(19) United States
(12) Patent Application Publication
(10) Pub. No.: US 2008/0171083 A1
(43) Pub. Date: Jul. 17, 2008

(54) Pharmaceutical Compositions Comprising an Amphiphilic Starch

(75) Inventors: John Staniforth, Wiltshire (GB); Naresh Talwar, Wiltshire (GB)

Correspondence Address: Davidson, Davidson & Kappel, LLC, 485 7th Avenue, 14th Floor, New York, NY, 10018, US

(73) Assignee: VECTURE LIMITED, WILTSHIRE (GB)

(21) Appl. No.: 11/578,271

(22) PCT Filed: Apr. 14, 2005
(86) PCT No.: PCT/GB2005/050051

§ 371 Date: Sep. 28, 2007

(30) Foreign Application Priority Data
Apr. 14, 2004 (GB) 0408308.5

(51) Int. Cl. A61K 9/64; A61K 9/20

(52) U.S. Cl. 424/456; 424/464

(57) ABSTRACT

The present invention relates to controlled or sustained release solid pharmaceutical compositions, to pharmaceutical excipients for use in the manufacture of such compositions and to methods of producing such compositions and excipients. The controlled or sustained release excipients include a release controlling excipient comprising an amphiphilic starch.

The present invention relates to controlled or sustained release solid pharmaceutical compositions, to pharmaceutical excipients for use in the manufacture of such compositions and to methods of producing such compositions and excipients.

Controlled or sustained release pharmaceutical compositions are designed to release an incorporated pharmaceutically active agent into a physiological environment over an extended period of time, or after a delay following administration.

For any particular pharmaceutically active agent, the range of plasma levels that is both efficacious and does not provoke significant or toxic side effects is known as the agent's therapeutic window or range. Shortly after a single dose of an active agent has been administered to a patient, its plasma concentration will reach a peak value and then quite rapidly decay, as the agent is metabolised and eliminated from the patient's body. However, if an agent is administered in a controlled or sustained release composition designed to release it over time, the plasma concentration of the agent can be maintained at an elevated and steady value for an extended period of time. By tailoring the rate at which the agent is released from the composition, its plasma concentration can also be held within a narrow range. Controlled or sustained release compositions, therefore, allow dosing intervals to be extended and their use reduces the risk of a drug's plasma level straying out of its therapeutic window. The extended dosing intervals achievable through use of sustained or controlled release compositions can allow dosing frequencies of once or twice a day and, hence, to greater patient compliance.

Sustained or controlled release compositions, in which the active agent is incorporated within a matrix that controls its release into a physiological environment, have been known for some considerable time. For example, U.S. Pat. No. 3,065,143 disclosed sustained release tablets comprising a cellulose derivative, exemplified by hydroxypropylmethyl cellulose, in 1962. Slow release preparations consisting of a water-soluble hydroxyalkyl cellulose and a higher aliphatic alcohol were proposed in 1975 in British Patent No. 1405088. European Patent Application No. 0251459 proposed solid controlled release pharmaceutical compositions, comprising a matrix of a water-soluble polydextrose or cyclodextrin and a higher fatty alcohol or polyalkylene glycol, in 1988. This document also disclosed compositions in which a cellulose derivative was substituted for the polydextrose or cyclodextrin.

Other materials, known to be suitable for providing a matrix for a sustained release pharmaceutical composition, include the acrylic polymers marketed under the trade name EUDRAGIT, polyglycolic acid, polylactic acid and copolymers of glycolic and lactic acid. The latter are often used in injectable or implantable compositions of the type disclosed in European Patent Application No. 0580428 and U.S. Pat. Nos. 4,954,298 and 5,061,492.

In other systems, the sustained or controlled release of a pharmaceutically active agent is achieved through the use of a release rate limiting coating applied to a core containing the active agent. One such system is described in European Patent Application No. 0147780, in which a core containing the active agent is coated with a film of polyvinyl alcohol, through which the active agent is gradually released when the device is inside the gastrointestinal tract.

Thus, it is clear that there are various approaches to controlling the release of an active agent from a dosage form. Where the matrix within which the active agent is dispersed is itself the release rate controlling element, it is generally accepted that the matrix cannot be formed solely from a material which is degraded in the body under physiological conditions. Such uncontrolled degradation of the excipient matrix would lead to “dumping” of the active agent, the majority of the dose being released quickly and as soon as the excipient degrades under physiological conditions. According to conventional wisdom, in order to avoid such uncontrolled dose dumping, the excipient or matrix must include at least one further component in addition to the degraded component. This additional component is required to control the release of the active agent and, usually, degradation or dispersion of the degraded component.

Indeed, where known controlled or sustained release compositions include a component which is degraded under physiological conditions, measures are always taken to reduce the breakdown of the first component, either in the form of a coating surrounding the degradable excipient, or in the form of a further excipient component which prevents or at least slows the degradation, usually by cross-linking with the degradable component, thereby retaining the degraded excipient component as part of the matrix for as long as possible.

It would be desirable to provide a simple, cheap and safe release rate controlling excipient, release from which is not affected by the changing physiological conditions between administration and delivery or release of the active agent.

In addition to the rate at which the active agent is released from the controlled or sustained release composition, the present invention seeks to provide an excipient which is suitable for carrying active agents with both wide and narrow absorption windows. The absorption window of an active agent is that part of the gastrointestinal tract from which the active agent is effectively and efficiently absorbed. Absorption windows vary greatly between active agents. Some active agents are well absorbed throughout the small intestine, for example propanolol hydrochloride and galantamine. In contrast, other active agents are only properly absorbed in specific parts of the small intestine. The main site of absorption of ciprofloxacin, for example, is the upper gastro-intestinal tract, up to the jejunum.

Thus, it is clearly desirable to control the release of the active agent from the dosage form so that absorption is maximised. This means that the excipient is preferably adapted to ensure that the active agent is primarily released in those parts of the gastro-intestinal where it is best absorbed. This should reduce wastage of the active agent, thereby increasing the effective dose achieved by administering a given amount of active agent.

In accordance with a first aspect of the present invention, there is provided a controlled or sustained release excipient comprising an amphiphilic starch as a release rate retarding component.

In a further aspect of the present invention, controlled or sustained release pharmaceutical compositions are provided, comprising an excipient according to the first aspect of the invention, and an active agent. Preferably, the active agent is uniformly dispersed throughout the controlled or sustained release excipient.

It has surprisingly been found that these amphiphilic starches can be used to form controlled or sustained release excipients, providing a unique matrix having both hydrophilic and lipophilic (amphiphilic) characteristics.

The use of the amphiphilic starch, which is degraded under physiological conditions, is surprisingly effective. It is totally unexpected that the excipient does not simply “dump” the dose of active agent upon administration, as one would anticipate, based upon the teaching in the prior art. Indeed, a person skilled in the technical field of sustained or controlled release excipients would not have considered amphiphilic starches to be suitable for controlling release of active agents dispersed therein. Rather, the breakdown of amphiphilic starches by amylase, despite their modification, would have meant that the skilled person would consider amphiphilic starches to be unable to control the release of an active agent. The use of an amphiphilic starch as the release rate controlling component has the advantage that it is not necessary to rely upon the interaction between two or more components in order to form a release rate controlling matrix. Such interactions are relied upon in known excipients which comprise components which are degraded under physiological conditions, as it is these interactions that control the breakdown of the excipient. It is undesirable to be reliant on such interactions, especially given the changing physiological conditions the excipients are exposed to upon ingestion. These changing conditions can affect the interactions between components of a complex excipient and this, in turn, can affect the release of the active agent.

Amphiphilic starch is modified starch which has a polar, water-soluble group and a non-polar, insoluble group. The starting material is a waxy starch slurry, which is easily derived from maize, and the like. The starch slurry is then treated with a substituted cyclic anhydride, for example substituted succinic or glutaric acid anhydrides. The resultant product, which has both hydrophilic and hydrophobic properties (amphiphilic), is then washed and dried.

A preferred amphiphilic starch for use in the present invention is alkenyl succinate starch. This chemically modified starch is produced by treating starch with alkenyl succinic anhydride under controlled pH conditions. In a preferred embodiment, the amphiphilic starches are prepared using n-octenyl succinic anhydride (n-OSA). The resultant starches are also referred to as OSA starches. The degree of substitution on these starch derivatives is around 3%. The OSA starches also have good compressibility and also allow good hardness of a tablet, making them suitable for pharmaceutical compressed tablet formulations.

The formation of octenyl alkenyl succinate starch is shown below.

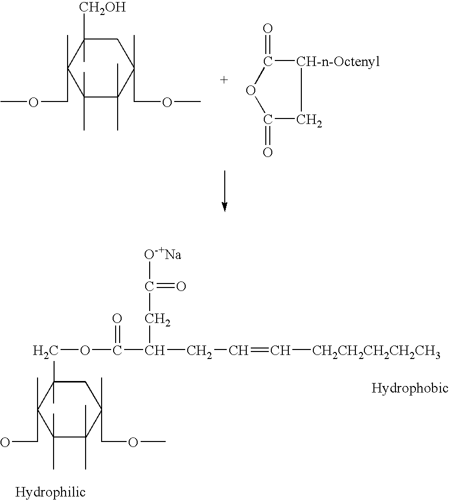

Alkenyl succinate starches are safe for human consumption and are used in the food and cosmetic industry as emulsifying and stabilising agents. These derivatives have been used in salad dressings, cakes, coffee whiteners, creamers and beverages, and in flavour emulsions as encapsulating agents.

In accordance with the present invention, amphiphilic starches are preferably alkenyl succinate starches, and more preferably octenyl succinate derivatives. These are marketed under the brand name C*EmTex by Cerestar, SA and as Capsul, Purity Gum and N-Creamer by National Starch Company. The C*EmTex 12638 product manufactured by Cerestar is an alkenyl succinate starch that is pregelatinised, stabilised waxy maize starch and is commonly known as starch sodium octenyl succinate. This starch is used as an emulsifying agent in dressings, sausages, processed cheese and coffee whiteners. The alkenyl succinate starch for use in the compositions and excipients in accordance with the present invention may also be synthesized using long chain fatty acids, the examples include C1618 alkenyl succinic anhydride, dodecenyl succinic anhydride, iso-butyl succinic anhydride, iso-octadecenyl succinic anhydride, n-decenyl succinic anhydride, n-dodecenyl succinic anhydride, n-hexadecenyl succinic anhydride, n-octadecenyl succinic anhydride, n-octenyl succinic anhydride, n-tetradecenyl succinic anhydride, nonenyl succinic anhydride, octenyl di-succinic acid and branched butenyl succinic anhydride.

The preferred amphiphilic starch used in accordance with the present invention is n-octenyl succinate starch.

The amphiphilic starch is the primary release rate controlling agent in the excipient according to the first aspect of the present invention. Preferably, the excipient does not include any other, conventional release rate controlling agents. In particular, the excipient does not include xanthan gum, a conventional sustained release excipient component. Also, the excipient of the present invention preferably does not include a polysaccharide. In a further embodiment, the excipient according to the present invention does not include an agent capable of cross-linking with the amphiphilic starch.

Amphiphilic starch is degraded upon ingestion by hydrolysis catalysed by the enzyme amylase. Amylase breaks down naturally occurring starch, such as that present in foodstuffs, by cleaving bonds between the glucose subunits. Although the starch used according to the present invention has been modified, the amylase is still able to act upon and degrade it.

Amylase is present in saliva and it starts to work on breaking down starch in food whilst it is being chewed in the oral cavity. Further amylase is secreted by the pancreas and works on degrading starch when the food leaves the stomach and enters the small intestine.

In the fed-state, that is, shortly after food has been ingested, the stomach will contain food and some amylase which accompanied the food into the stomach following mastication. In this state there will be a low level of amylase activity within the stomach, although this activity will be restricted by the presence of the stomach acid, which inhibits the enzyme's activity. The amylase activity in the stomach will be negligible in the fasted-state, that is, when there is little or no food in the stomach. In this state there will be little or no amylase present in the stomach.

When a tablet, capsule or other dosage form is swallowed by a patient, very little saliva is swallowed with it. The secretion of saliva into the oral cavity is generally triggered by chewing of food and the saliva is then swallowed with the food and travels with the food into the stomach. Therefore, if a dosage form is swallowed when the patient is in the fasted state, a negligible amount of saliva will be swallowed at the same time. What is more, there will be little or no amylase activity in the stomach and so the dosage form will not really be exposed to amylase until it reaches the small intestine.

Amylase degradation of starch can be prevented, at least temporarily, by an enzyme activity reducing agent or an enzyme inhibitor. Preferably the enzyme inhibitor is an amylase inhibitor. Amylase activity is inhibited by low pH. Therefore, according activity reducing agent such as citric acid, succinic acid, tartaric acid, fumaric acid, maleic acid, lactic acid, ascorbic acid, sodium dihydrogen phosphate, potassium dihydrogen phosphate and the like. Alternatively, the composition may include an enzyme inhibitor such as ascorbic acid, acarbose, phaseolamine, tendaminstat, maltose, maltotriose and nojirimycin.

However, it is important to note acids which act as enzyme activity reducing agents are not compatible with all active agents that one might disperse within the excipients according to the present invention. It has been found that some active agents are unstable in the presence of acid over an extended period of time. This means that such active agents cannot be included in compositions which include an acid. Examples of such “acid-sensitive” active agents are given below, and they include gabapentin and galantamine.

As alluded to above, another aspect of the invention is the formulation of gastric-retained controlled release excipients. Surprisingly, the excipients and compositions described in the present invention have the property of floating in aqueous fluids. Such excipients and compositions are therefore suitable for carrying and dispensing active agents which have an absorption window such that they are predominantly from upper parts of the gastrointestinal tract. The gastric-retained or hydrodynamically balanced delivery systems are used to retain the dosage form for prolonged periods in the stomach, thus improving the retention time of the dosage form in the upper part or start of the small intestine, where many active agents with narrow absorption windows are preferably absorbed. The following active agents have narrow absorption windows and they are best absorbed in the upper parts of the gastro-intestinal tract: ciprofloxacin, gabapentin, ranitidine, cefaclor, acyclovir, cyclosporin, benazepril, ferrous sulfate and cephalexin.

These active agents may be formulated with or without an enzyme activity reducing agent (such as citric acid) so as to reduce amylase attack on the excipient matrix. Where the active agent is only absorbed from the upper parts of the small intestine, the composition preferably does not include an enzyme activity reducing agent.

The buoyancy of the excipients according to the present invention is good. However, the buoyancy can be improved by the addition of gas generating agents. The gas generating agents react with the aqueous contents of the stomach to generate a gas, preferably carbon dioxide. The gas gets entrapped in the matrix and allows the dosage form to float. Examples of gas generating agents include carbonates like sodium carbonate, sodium bicarbonate, calcium carbonate, sodium glycine carbonate, potassium bicarbonate, sulfites like sodium sulfite, sodium metabisulfite and the like. These gas generating agents evolve gas upon reaction with acid. This acid can be the acid present in the stomach. Alternatively, the acid may be included in the composition, as discussed above. Acids suitable for inclusion as part of an effervescent gas generating couple include citric acid, malic acid, fumaric acid, tartaric and the like, and their salts.

As mentioned above, some active agents are unstable in the presence of an acid over an extended period of time, and such active agents should not be administered in excipients which include an acid. In this case, the excipient may still include a gas generating agent which will react with the acid in the stomach, in order to enhance buoyancy.

Where the active agent to be administered is (a) unstable in a composition which includes an acid and (b) is preferably absorbed in the upper gastro-intestinal tract, this active agent is preferably administered in an excipient which does not include an acid but which does include a gas generating agent which will react with the acid in the stomach to generate gas and increase the buoyancy of the dosage form.

Where the active agent to be administered is (a) stable in an excipient which includes an acid and (b) is preferably absorbed in the upper gastro-intestinal tract, this active agent can be administered in an excipient which includes an acid and a gas generating agent which will react with that acid to generate gas and increase the buoyancy of the dosage form. Alternatively, such an active agent can be administered in an acid-free excipient which includes a gas generating agent which will react with the acid in the stomach.

Where the active agent has a wide absorption window, gastro-retention of the dosage form is not so significant, and the gas generating agent can be omitted without significant loss of absorption. Nevertheless, it may remain desirable to include the acid as an amylase inhibitor, provided it is compatible with the active agent in question. Examples of active agents which have a wide absorption window and which are absorbed throughout the gastrointestinal tract include: propranolol, diltiazem, nifedipine, pseudoephedrine, diclofenac, metoprolol, galantamine, chlorpheniramine and ephedrine. These active agents are preferably formulated with an enzyme activity reducing agent, so as to prevent rapid release of the active agent in the presence of amylase.

Where the active agent has a wide absorption window but is unstable in an excipient which includes an acid, absorption can maximised by using a composition comprising a low proportion of active agent and a high proportion of amphiphilic starch. In such a composition, the increased proportion of amphiphilic starch present means that the enzyme must degrade more of the excipient in order to release the active agent dispersed therein. In such an embodiment, the active agent is preferably still uniformly dispersed within the excipient. Degradation of the amphiphilic starch takes longer and so the active agent is released more gradually. Such an excipient is suitable for administering galantamine.

The present invention further provides controlled or sustained release excipients and compositions further comprising hydrophobic materials, along with the release-retarding amphiphilic starch. The inclusion of a fatty or oily component slows the hydration of the starch molecules and consequently the viscosity development, thus allowing the slower erosion of the starch matrix resulting in better release retarding efficacy.

Examples of the types of hydrophobic material which may be included in the excipients and compositions according to the present invention include fatty or oily materials, such as vegetable oils and, in particular, hydrogenated vegetable oils. The hydrogenated oils include the type 1 and 2 oils as per the United States Pharmacopocial specifications, the most preferred ones are hydrogenated cottonseed oil, hydrogenated castor oil, hydrogenated palm oil and hydrogenated soybean oil. The examples of other hydrophobic substances that may be employed in the present invention include sodium stearyl fumarate, calcium stearate, magnesium stearate, glyceryl monooleate, glyceryl monostearate, glyceryl palmitostearate, medium chain glycerides, mineral oil and stearyl alcohol.

It is envisaged that a plurality of oil or fatty components can be included in the excipients and compositions in accordance with the present invention. According to the present invention the oily or fatty components may be present up to 30%, 35%, 40%, 45% or 50% of the alkenyl succinate starch content.

Conventional compositions containing oily or fatty substances generally suffer from the disadvantage that the erosion of the matrix is reduced, leading to longer diffusion path lengths for the drugs and resulting in slower terminal release rates. This means that it is not possible to obtain a near zero-order release using a composition including a hydrophobic component.

The co-processed materials of the present invention do not suffer from this problem and exhibit nearly constant release of the active ingredient. This effect is due to the presence of the amphiphilic starch which has the property of erosion. Thus, combinations of amphiphilic starch and hydrophobic material can be used as excipients for formulating controlled-release compositions of a variety of drugs. The starch may be present up to 75%, 70%, 65%, 60%, 55% or 50% of the total weight of the composition.

A significant advantage enjoyed by embodiments of the above described aspects of the invention is that they can include in excess of 50% and, preferably, in excess of 60, 70 or 80% active agent or drug.

Additional protection from amylase and other chemicals in the stomach may be required to fine-tune the release of the active agent from an excipient or composition according to the present invention. In one embodiment, the composition may have an enteric coating which protects the excipient and the active agents until the coating itself is broken down, preferably in a predetermined part of the gastrointestinal tract. Coatings of this type are well known and widely used. Examples of suitable materials for such coatings include polyvinyl alcohol, a polyacrylate, a polymethacrylate, a cellulose or a cellulose derivative, or a polymerised unsaturated fatty acid or derivative thereof.

A significant advantage of excipients in accordance with the invention is that they can be compressible and, thus, can be employed in a simple admixture with an active agent to prepare sustained release tablets by direct compression or, if desired, by wet or dry granulation. The fact that the excipient compositions in accordance with the invention can be provided in the form of dry and free flowing powders or granules renders them particularly suited to use in the preparation of tablets by direct compression techniques. Tablets formed using a composition in accordance with the present invention can enjoy all of the advantages associated with controlled or sustained release compositions in accordance with the invention, depending upon their exact formulation.

Solid pharmaceutical compositions in accordance with the present invention can be in the form of tablets, an extrudate, pellets, powders (for example, for nasal administration or inhalation), granules and suppositories (rectal and vaginal). Pharmaceutical compositions in accordance with the invention are preferably in the form of tablets for oral administration, including buccal and sublingual tablets. The most preferred for is tablets intended for ingestion and capable of releasing active agent over an extended period of time into the gastrointestinal tract.

The compositions and excipients according to the present invention are preferably sufficiently compressible that they can be simply mixed with an active agent, to form a sustained or controlled release tablet. It is envisaged that such tablets can be prepared by the direct compression of a mixture of active agent and excipient, or by the compression of a granulation formed by wet or dry granulating the excipient with an active agent. The tablets may be subsequently coated.

The tablets can include additional pharmaceutical excipients of a conventional nature including, for example, lubricants and glidants, binders, disintegrating agents, colouring agents, flavouring agents, bulking agents, fillers, preservatives and stabilizers, as appropriate.

A capsule can be manufactured, filled with a composition according to the present invention, comprising the excipient including amphiphilic starch and any other appropriate excipient components, and an active agent.

Binders suitable for use in excipients and compositions according to the present invention include microcrystalline cellulose, gelatin, polyvinyl pyrrollidone, acacia, alginic acid, guar gum, hydroxypropyl methylcellulose, sucrose and polyethylene oxide.

According to the present invention the alkenyl succinate starch may also be used as a binder and granulating agent.

Lubricants and glidants include talc, magnesium stearate, calcium stearate, stearic acid, zinc stearate, glyceryl behenate, sodium stearyl fumarate and silicon dioxide.

Preferably, fillers and bulking agents for use in the excipients and compositions of the present invention include dicalcium phosphate, microcrystalline cellulose, starch, calcium sulfate, lactose, kaolin, mannitol, sodium chloride, calcium carbonate, dextrates, dextrin, dextrose, sorbitol and sucrose.

As suggested above, the most preferred form of pharmaceutical compositions in accordance with the present invention is a tablet intended for ingestion and capable of releasing an active agent into the gastrointestinal tract over an extended period of time. It is preferred that such tablets are formulated to release their payload over a period which allows once daily dosing. This period will vary depending upon the properties of the active agent. For example, it can be advantageous for the serum concentration of certain active agents to fall below a given threshold for a period of mononitrate and isosorbide dinitrate) and for these to be released over shorter periods of time than others.

It is preferred that the composition comprises in excess of 50% and, preferably, in excess of 60, 70 or 80% active agent by weight. Preferably, the active agent is dispersed throughout the excipient, for gradual release as the excipient degrades or disintegrates.

Classes of drugs which are suitable in the present invention include antacids, anti-inflammatory substances, coronary dilators, cerebral dilators, peripheral vasodilators, anti-infectives, psychotropics, anti-manics, stimulants, anti-histamines, laxatives, decongestants, vitamins, gastro-intestinal sedatives, anti-diarrheal preparations, anti-anginal drugs, vasodilators, anti-arrhythmics, anti-hypertensive drugs, vasoconstrictors and migraine treatments, anti-coagulants and anti-thrombotic drugs, analgesics, anti-pyretics, hypnotics, sedatives, anti-emetics, anti-nauseants, anti-convulsants, neuromuscular drugs, hyper- and hypoglycemic agents, thyroid and anti-thyroid preparations, diuretics, anti-spasmodics, uterine relaxants, mineral and nutritional additives, anti-obesity drugs, anabolic drugs, erythropoietic drugs, anti-asthmatics, bronchodilators, expectorants, cough suppressants, mucolytics, drugs affecting calcification and bone turnover and anti-uricemic drugs.

Specific drugs or active agents that can be incorporated into compositions in accordance with the present invention include gastro-intestinal sedatives such as metoclopramide and propantheline bromide; antacids such as aluminum trisilicate, aluminum hydroxide, ranitidine and cimetidine; anti-inflammatory drugs such as phenylbutazone, indomethacin, naproxen, ibuprofen, flurbiprofen, diclofenac, dexamethasone, prednisone and prednisolone; coronary vasodilator drugs such as glyceryl trinitrate, isosorbide dinitrate and pentaerythritol tetranitrate; peripheral and cerebral vasodilators such as soloctidilum, vincamine, naftidrofuryl oxalate, co-dergocrine mesylate, cyclandelate, papaverine and nicotinic acid; anti-infective substances such as erythromycin stearate, cephalexin, nalidixic acid, tetracycline hydrochloride, ampicillin, flucloxacillin sodium, hexamine mandelate and hexamine hippurate; neuroleptic drugs such as flurazepam, diazepam, temazepam, amitryptyline, doxepin, lithium carbonate, lithium sulfate, chlorpromazine, thioridazine, trifluperazine, fluphenazine, piperothiazine, haloperidol, maprotiline hydrochloride, imipramine and desmethylimipramine; central nervous stimulants such as methylphenidate, ephedrine, epinephrine, isoproterenol, amphetamine sulfate and amphetamine hydrochloride; antihistamic drugs such as diphenhydramine, diphenylpyraline, chlorpheniramine and brompheniramine; anti-diarrheal drugs such as bisacodyl and magnesium hydroxide; the laxative drug, dioctyl sodium sulfosuccinate; nutritional supplements such as ascorbic acid, alpha tocopherol, thiamine and pyridoxine; anti-spasmodic drugs such as dicyclomine and diphenoxylate; drugs affecting the rhythm of the heart such as verapamil, nifedipine, diltiazem, procainamide, disopyramide, bretylium tosylate, quinidine sulfate and quinidine gluconate; drugs used in the treatment of hypertension such as propranolol hydrochloride, guanethidine monosulphate, methyldopa, oxprenolol hydrochloride, captopril and hydralazine; drugs used in the treatment of migraine such as ergotamine; drugs affecting coagulability of blood such as epsilon aminocaproic acid and protamine sulfate; analgesic drugs such as acetylsalicylic acid, acetaminophen, codeine phosphate, codeine sulfate, oxycodone, dihydrocodeine tartrate, oxycodeinone, morphine, heroin, nalbuphine, butorphanol tartrate, pentazocine hydrochloride, cyclazacine, pethidine, buprenorphine, scopolamine and mefenamic acid; anti-epileptic drugs such as phenyloin sodium and sodium valproate; neuromuscular drugs such as dantrolene sodium; substances used in the treatment of diabetes such as tolbutamide, disbenase glucagon and insulin; drugs used in the treatment of thyroid gland dysfunction such as triiodothyronine, thyroxine and propylthiouracil, diuretic drugs such as furosemide, chlorthalidone, hydrochlorthiazide, spironolactone and triamterene; the uterine relaxant drug ritodrine; appetite suppressants such as fenfluramine hydrochloride, phentermine and diethylproprion hydrochloride; anti-asthmatic and bronchodilator drugs such as aminophylline, theophylline, salbutamol, orciprenaline sulphate and terbutaline sulphate; expectorant drugs such as guaiphenesin; cough suppressants such as dextromethorphan and noscapine; mucolytic drugs such as carbocisteine; anti-septics such as cetylpyridinium chloride, tyrothricin and chlorhexidine; decongestant drugs such as phenylpropanolamine and pseudoephedrine; hypnotic drugs such as dichloralphenazone and nitrazepam; anti-nauseant drugs such as promethazine theoclate; haemopoietic drugs such as ferrous sulphate, folic acid and calcium gluconate; uricosuric drugs such as sulphinpyrazone, allopurinol and probenecid; calcification affecting agents such as biphosphonates, e.g., etidronate, pamidronate, alendronate, residronate, teludronate, clodronate and alondronate; and anti-alzheimers drugs, such as acetylcholinesterase inhibitors like donezepil, rivastigmine, tacrine and galantamine.

More drugs or active agents which are candidates for incorporation into compositions in accordance with the invention include, but are not limited to, H₂ receptor antagonists, antibiotics, analgesics, cardiovascular agents, peptides or proteins, hormones, anti-migraine agents, anti-coagulant agents, anti-emetic agents, anti-hypertensive agents, narcotic antagonists, chelating agents, anti-anginal agents, chemotherapy agents, sedatives, anti-neoplastics, prostaglandins, antidiuretic agents and the like. Typical drugs include but are not limited to nizatidine, cimetidine, ranitidine, famotidine, roxatidine, etinidine, lupitidine, nifentidine, niperitone, sulfotidine, tuvatidine, zaltidine, erythomycin, penicillin, ampicillin, roxithromycin, clarithromycin, psylium, ciprofloxacin, theophylline, nifedipine, prednisone, prednisolone, ketoprofen, acetaminophen, ibuprofen, dexibuprofen lysinate, flurbiprofen, naproxen, codeine, morphine, sodium diclofenac, acetylsalicylic acid, caffeine, pseudoephedrine, phenylpropanolamine, diphenhydramine, chlorpheniramine, dextromethorphan, berberine, loperamide, mefenamic acid, flufenamic acid, astemizole, terfenadine, certirizine, phenyloin, guafenesin, N-acetylprocainamide HCl, pharmaceutically acceptable salts thereof and derivatives thereof. Other agents include antibiotics such as clarithromycin, amoxicillin erythromycin, ampicillin, penicillin, cephalosporins, e.g., cephalexin, pharmaceutically acceptable salts thereof and derivatives thereof, acetaminophen and NSAIDS such as ibuprofen, indomethacin, aspirin, diclofenac and pharmaceutically acceptable salts thereof.

The most preferred active agents are gabapentin, galantamine, topiramate, oxycodone, oxymorphone, hydromorphone and methylphenidate.

Both pharmaceutical compositions and excipients in accordance with the invention can include a water soluble channeling agent. The latter is selected to facilitate the penetration of water from a physiological environment into the composition (or into a pharmaceutical composition formed from the excipient), or the egress of active agent from the composition (or from a pharmaceutical composition formed from the excipient) into a physiological environment. Suitable channeling agents include inorganic salts such as sodium chloride, sugars such as dextrose, sucrose, mannitol, xylitol, and lactose, and water soluble polymers such as polyvinylpyrrolidone and polyethyleneglycols.

The invention extends to compositions whenever prepared by employing an excipient in accordance with the invention, or by one of the above discussed methods in accordance with the invention. Such methods can involve a final step in which a coating is applied to the composition in order to provide a final dosage form. The coating can be of a conventional nature, for example it can comprise polyvinyl alcohol, a polyacrylate, a polymethacrylate, or a cellulose or a cellulose derivative, or it can be formed from a polymerised unsaturated fatty acid or derivative of the nature employed in previously described aspects of the invention. The coating is preferably unbroken and can be capable of resisting penetration by stomach acid.

An advantage of any aspect or embodiment of the invention that includes a coating is that it allows the food effect, which can be particularly problematic with tablets which have a high oil content, to be avoided.

The compositions according to the present invention may be made into dosage forms in a number of ways. Firstly, the active agent and the excipient, for example, alkenyl succinate starch, are dry blended along with lubricants and optionally diluents and compressed directly into a tablet or the dry powder blend is filled in a capsule shell to achieve controlled or sustained release of the active agent. The alkenyl starch may also be processed by granulating it with an alcoholic or a hydro-alcoholic solvent in order to obtain granules having better flow as compared to a dry blend.

In a second embodiment, a powder blend of the alkenyl succinate starch and the active agent are wet-granulated with an aqueous, alcoholic or a hydro-alcoholic solvent and dried below 80° C. The dried granules are then mixed with lubricants and optionally diluents and compressed into tablets or filled in capsules. Surprisingly, the tablets formed using wet granulation exhibit better release control than the tablets formed by addition as a dry powder as described above. The flow properties of the granules are also improved.

In a third embodiment, alkenyl succinate starch is dry blended or co-processed with an oily or fatty material to form an excipient comprising an amphiphilic starch and a hydrophobic component. The co-processed materials exhibit improved flow properties of the granules as compared to the dry blends. The co-processing may be done by granulation with an aqueous, alcoholic or a hydro-alcoholic solvent. The co-processing can also be performed in the presence of an active agent.

The following examples are provided merely to illustrate the various aspects of the invention and to assist in their understanding. They should not be construed as in any way limiting the scope of the present invention.

The examples cover all four classes of molecules as described by the USFDA's Biopharmaceutics Classification System (BCS).

### EXAMPLE 1

This example illustrates a controlled release composition containing Indomethacin (a class-2 drug, highly permeable, low solubility) as an active and starch sodium octenyl succinate as a release controlling agent. The composition is illustrated in Table 1.

TABLE 1

| Ingredients | mg/tab |
|---|---|
| Indomethacin | 50 |
| Starch sodium octenyl succinate | 397.50 |
| (C*Emtex 12638, supplied by Cerestar, UK) | |
| Calcium stearate | 2.5 |

The method comprised the following steps:
1. Indomethacin and starch sodium octenyl succinate were screened through 850 micron mesh.
2. Calcium stearate was screened through 355 micron mesh.
3. Powders of step-1 and 2 were mixed.
4. Tablets were compressed using 11 mm round tooling.

The tablets were tested for dissolution in USP-1 apparatus, the basket speed was 100 rpm and the media employed was 900 ml of phosphate buffer pH 6.8. Dissolution results are shown in Table 2.

TABLE 2

| Time (hours) | % drug dissolved |
|---|---|
| 1 | 5 |
| 2 | 13 |
| 4 | 30 |
| 6 | 46 |
| 8 | 61 |
| 10 | 81 |
| 12 | 95 |

### EXAMPLE 2

This example illustrates a controlled release composition containing Gabapentin (a class-3 drug, low permeability, high solubility) as an active molecule and starch sodium octenyl succinate as a release controlling agent. Tablets were compressed directly. The composition is illustrated in Table 3.

TABLE 3

| Ingredients | mg/tab |
|---|---|
| Gabapentin | 149.17 |
| Starch sodium octenyl succinate | 298.33 |
| (C*Emtex 12638) | |
| Emcocel 90M | 50 |
| Calcium stearate | 2.5 |

The method comprised the following steps:
1. Gabapentin, starch sodium octenyl succinate and Emcocel were passed through 850 micron mesh.
2. Calcium stearate was screened through 355 micron mesh
3. Powders of step 1 and 2 were mixed together.
4. Tablets were compressed using 11 mm round concave punches.

The tablets were tested for dissolution using the method as described in Example 1. The results are shown in Table 4.

TABLE 4

| Time (hours) | % drug dissolved |
|---|---|
| 1.0 | 30 |
| 2.0 | 47 |
| 4.0 | 71 |
| 6.0 | 92 |
| 8.0 | 100 |

### EXAMPLE 3

This example illustrates controlled-release formulations of Gabapentin manufactured by using starch sodium octenyl succinate and a combination with Sterotex-K (hydrogenated soybean and hydrogenated castor oil) as release controlling agent. The make up of the compositions are set out in Table 5. In these examples Gabapentin was granulated with starch sodium octenyl succinate to improve its flow and compression properties.

TABLE 5

| | Formulation A | Formulation B |
|---|---|---|
| Ingredients | (mg/tab) | (mg/tab) |
| Gabapentin | 225 | 225 |
| Starch sodium octenyl | 225 | 180 |
| succinate (C*Emtex 12638) | | |
| Sterotex-K (supplied by | — | 45 |
| Abitec Corp., USA) | | |
| Emcocel 90M | 47.5 | 47.5 |
| Calcium stearate | 2.5 | 2.5 |

The method comprised the following steps:
1. Gabapentin was granulated with starch sodium octenyl succinate paste (9% w/w in isopropyl alcohol:water mixture, 25:75).
2. Granules were screened through 850 micron mesh and dried in a tray drier at 60° C.
3. Extragranular starch sodium octenyl succinate, Sterotex K and Emcocel 90M were screened through 850 micron mesh and calcium stearate was passed through 250 micron mesh.
4. Powders of step 2 and 3 were blended together.
5. Tablets were compressed using 11 mm round concave punches.

Tablets were tested for dissolution as described in Example 1 and the results are recorded in Table 6.

TABLE 6

% drug dissolved

| Time (hours) | Formulation A | Formulation B |
|---|---|---|
| 1 | 33 | 25 |
| 2 | 55 | 40 |
| 4 | 90 | 58 |
| 6 | 96 | 73 |
| 8 | — | 83 |
| 10 | — | 92 |

### EXAMPLE 4

This example illustrates a controlled release tablet of Gabapentin formulated using wet granulation of a mix of Gabapentin and starch sodium octenyl succinate with a solvent system containing water and isopropyl alcohol. Table 7 shows the make up of the composition.

TABLE 7

| Ingredients | mg/tab |
|---|---|
| Gabapentin | 250 |
| Starch sodium octenyl succinate (C*Emtex 12638) | 197.5 |
| Emcocel 90 M | 50 |
| Calcium stearate | 2.5 |

The method comprised the following steps:
1. Gabapentin and starch sodium octenyl succinate were weighed and blended together.
2. The blend was granulated with water:isopropyl alcohol mixture (60:40)
3. Granules were tray dried at 60° C. for 30 minutes.
4. The granules were passed through 850 micron mesh and blended with calcium stearate (passed through 250 micron mesh)
5. Tablets were compressed using 11 mm round standard concave punches.

Tablets were tested for dissolution as described in Example 1. The results of the dissolution tests are set out in Table 8.

TABLE 8

| Time (hours) | % drug dissolved |
|---|---|
| 1 | 27 |
| 2 | 42 |
| 4 | 65 |
| 6 | 82 |
| 8 | 95 |
| 10 | 98 |

### EXAMPLE 5

This example illustrates a capsule-based controlled release formulation using starch sodium octenyl succinate as release controlling agent. The make up of the composition is set out in Table 9.

TABLE 9

| Ingredients | mg/capsule |
|---|---|
| Indomethacin | 50 |
| Starch sodium octenyl succinate | 310 |

The method comprised the following steps:
1. Indomethacin and starch sodium octenyl succinate were passed through 850 micron mesh and blended together.
2. Blend was filled in size ‘0’ gelatin capsules. The target fill weight was 360 mg.

Capsules were tested for dissolution using USP apparatus 2, the paddle height being 4.5 cm, baskets were used as sinkers and 900 ml of pH 6.8 phosphate buffer was used as a dissolution media. Results of the dissolution test are recorded in Table 10.

TABLE 10

| Time (hours) | % drug dissolved |
|---|---|
| 1 | 18 |
| 2 | 36 |
| 4 | 66 |
| 6 | 96 |
| 8 | 105 |

### EXAMPLE 6

This example illustrates the formulation of hydrodynamically balanced tablets of Gabapentin. The composition make up is set out in Table 11.

TABLE 11

| Ingredients | mg/tab |
|---|---|
| Gabapentin | 250 |
| Starch sodium octenyl succinate | 197.5 |
| Calcium carbonate | 200 |
| Calcium stearate | 2.5 |

The method comprised the following steps:
1. Gabapentin and starch sodium octenyl succinate were passed through 850 micron mesh and blended together.
2. The powder of step 1 was granulated with isopropyl alcohol, water mixture in a ratio of 60:40.
3. The granules were tray dried at 60° C. for 30 minutes.
4. Dried granules were passed through 850 micron mesh.
5. Calcium carbonate and calcium stearate (passed through 355 micron mesh) were mixed to the granules of step-4 and compressed into tablets using 11 mm round, standard concave punches.

The tablets were tested for dissolution using USP type 1 dissolution apparatus using 900 ml of 0.1N HCl as a dissolution media. The basket speed was 100 rpm. The results are shown in Table 12.

TABLE 12

| Time (hours) | % drug dissolved |
|---|---|
| 1 | 23 |
| 2 | 37 |
| 4 | 56 |
| 6 | 71 |
| 8 | 85 |
| 10 | 89 |
| 12 | 91 |

The tablets were tested for buoyancy using USP-2 apparatus, at a paddle speed of 25 rpm using 900 ml 0.1N HCl as media. The tablets achieved buoyancy in 30 minutes and remained floating at the top of the media thereafter.

### EXAMPLE 7

This example illustrates the formulation of controlled release Gabapentin tablets by 2 different methods (a) by granulation together of starch sodium octenyl succinate with drug (b) and direct compression of drug and starch sodium octenyl succinate. Both the methods had similar composition. Table 13 shows the make up of the composition.

TABLE 13

| Ingredients | mg/tab |
|---|---|
| Gabapentin | 300 |
| Starch sodium octenyl succinate | 393 |
| Calcium stearate | 7 |

Method (a) comprised the steps of:
1. Gabapentin and starch sodium octenyl succinate were passed through 850 micron mesh.
2. The powder of step 1 was granulated with isopropyl alcohol, water mixture in a ratio of 60:40.
3. Granules were dried at 60° C. in a tray drier.
4. The dried granules were passed through 850 micron mesh and were blended with calcium stearate (passed through 250 micron mesh)
5. Tablets were compressed using 11 mm, round, standard concave punches.

Method (b) comprised the steps of:
1. Gabapentin and starch sodium octenyl succinate were passed through 850 micron mesh.
2. Calcium stearate was passed through 250 micron mesh.
3. Powders of step-1 and 2 were blended together.
4. Tablets were compressed using 11 mm round standard concave punches.

Results of the dissolution tests are set out in Table 14.

TABLE 14

% drug dissolved

| Time (hours) | Method (a) | Method (b) |
|---|---|---|
| 1 | 22 | 25 |
| 2 | 33 | 40 |
| 4 | 51 | 64 |
| 6 | 65 | 80 |
| 8 | 77 | 93 |
| 10 | 84 | 101 |
| 12 | 87 | — |

### EXAMPLE 8

The present example illustrates the formulation of an excipient comprising of starch sodium octenyl succinate and sterotex-NF (supplied by Abitec Corp. USA).

The method comprised the following steps:
1. Starch sodium octenyl succinate and sterotex-NF in the ratio of 80 and 20 were blended together.
2. Blend of step-1 was granulated with isopropyl alcohol, water mixture in 90:10 ratio.
3. Granules were dried at 60° C. for 30 minutes.
4. Dried granules were passed through 850 micron mesh.

### EXAMPLE 9

This example illustrates the formulation of controlled release tablets of Gabapentin using the excipient of Example 8. Table 15 shows the make up of the composition.

TABLE 15

| Ingredients | mg/tab |
|---|---|
| Gabapentin | 300 |
| Excipient of Example 8 | 393 |
| Calcium stearate | 7 |

The tablets were compressed as described in Example 7. The results of the dissolution tests are recorded in Table 16.

TABLE 16

| Time (hours) | % drug dissolved |
|---|---|
| 1 | 27 |
| 2 | 38 |
| 4 | 51 |
| 6 | 62 |
| 8 | 71 |
| 10 | 78 |
| 12 | 82 |

### EXAMPLE 10

This example illustrates the formulation of an excipient based on the processing of starch sodium octenyl succinate by wet granulation. Processing is found to improve the flow properties of the granules and their compression characteristics.

The method comprised the following steps:
1. Starch sodium octenyl succinate was passed through 850 micron mesh.
2. The powder was granulated with the mixture of isopropyl alcohol and water (90:10)
3. Granules were tray dried at 60° C. and screened through 850 micron mesh to obtain the excipient.

### EXAMPLE 11

This example illustrates the controlled release tablet of Gabapentin using the excipient of Example 10. Table 17 shows the make up of the composition.

TABLE 17

| Ingredients | mg/tab |
|---|---|
| Gabapentin | 300 |
| Excipient of Example 10 | 393 |
| Calcium stearate | 7 |

The method comprised the following steps:
1. Gabapentin and the excipient were passed through 850 micron mesh.
2. Calcium stearate was passed through 355 micron mesh.
3. Powders of step 1 and 2 were mixed together.
4. Tablets were compressed using 11 mm tooling.

Dissolution tests were performed as described in Example 1, the results of which are recorded in Table 18.

TABLE 18

| Time (hours) | % drug dissolved |
|---|---|
| 1 | 26 |
| 2 | 39 |
| 4 | 60 |
| 6 | 77 |
| 8 | 90 |
| 10 | 91 |

### EXAMPLE 12

This example illustrates a sustained-release tablet formulation of propranolol hydrochloride (a class-1 drug; high solubility and high permeability) using starch sodium octenyl succinate as a release retarding agent. The make up of the composition is set out in Table 19.

TABLE 19

| Ingredients | mg/tab |
|---|---|
| Propranolol hydrochloride | 120 |
| Starch sodium octenyl succinate | 240 |
| (intragranular) | |
| Starch sodium octenyl succinate | 85 |
| (extragranular) | |
| Calcium stearate | 5 |

The method comprised the following steps:
1. Propranolol hydrochloride and starch sodium octenyl succinate (intragranular) were passed through 850 micron mesh and blended together.
2. Powder was granulated with water and isopropyl alcohol mixture of 20:80 ratio.
3. Granules were dried at 60° C. in a tray drier.
4. The dried granules were mixed with extragranular starch and calcium stearate, screened through 355 micron mesh and blended together.
5. Tablets were compressed using 11 mm round punches.

Tablets were tested for dissolution using USP-1 apparatus, basket speed of 100 rpm and using 900 ml of 0.1N HCl as a dissolution media. Results are recorded in Table 20.

TABLE 20

| Time (hours) | % drug dissolved |
|---|---|
| 1 | 19 |
| 2 | 33 |
| 4 | 57 |
| 6 | 82 |
| 10 | 88 |

### EXAMPLE 13

This example illustrates the formulation of sustained-release tablet formulation of propranolol hydrochloride using an excipient of Example 8. Table 21 shows the make up of the composition.

TABLE 21

| Ingredients | mg/tab |
|---|---|
| Propranolol hydrochloride | 120 |
| Excipient of Example 8 | 375 |
| Calcium stearate | 5 |

The method comprised the following steps:
1. Propranolol hydrochloride and the excipient were passed through 850 micron mesh and mixed together.
2. Calcium stearate was screened through 355 micron mesh and blended with the powder of step 1.
3. Tablets were compressed using 11 mm round punches.

The tablets were tested for dissolution as described in Example 12. The results are recorded in Table 22.

TABLE 22

| Time (hours) | % drug dissolved |
|---|---|
| 1 | 23 |
| 2 | 33 |
| 4 | 50 |
| 6 | 67 |
| 10 | 92 |

### EXAMPLE 14

This example illustrates a sustained-release formulation of propranolol using a mixture of starch sodium octenyl succinate and Sterotex-NF. The make up of the composition is shown in Table 23.

TABLE 23

| Ingredients | mg/tab |
|---|---|
| Propranolol hydrochloride | 120 |
| Starch sodium octenyl succinate | 240 |
| Sterotex NF | 85 |
| Calcium stearate | 5 |

The method comprised the following steps:
1. Propranolol hydrochloride and starch sodium octenyl succinate were passed through 850 micron mesh and blended together.
2. The powder was granulated with a solvent mixture of water and iso-propyl alcohol in ratio of 20:80.
3. Granules were dried at 60° C. in a tray drier.
4. Dried granules were mixed with sterotex NF and calcium stearate (screened through 355 micron mesh).
5. Tablets were compressed using 11 mm round punches.

The resultant tablets were tested for dissolution as described in Example 12 and the results of the tests are recorded in Table 24.

TABLE 24

| Time (hours) | % drug dissolved |
|---|---|
| 1 | 19 |
| 2 | 29 |
| 4 | 46 |
| 6 | 60 |
| 10 | 79 |
| 12 | 86 |

### EXAMPLE 15

This example illustrates a sustained release tablet formulation of a class-4 drug, Carvedilol (low solubility and low permeability). The make up of the composition is set out in Table 25.

TABLE 25

| Ingredients | mg/tab |
|---|---|
| Carvedilol | 50 |
| Starch sodium octenyl succinate | 150 |
| Emcocel 90 M | 196 |
| Calcium stearate | 4 |

The method comprised the following steps:
1. Starch sodium octenyl succinate, carvedilol and Emcocel 90M were passed through 850 micron mesh.
2. Calcium stearate was passed through 250 micron mesh.
3. The powders of step 1 and 2 were blended and tablets were compressed using 11 mm punches.

The tablets were tested for dissolution using a media containing 1% sodium lauryl sulphate in 0.1N HCl, USP 1 apparatus, basket speed 100 rpm. Results of the tests are recorded in Table 26.

TABLE 26

| Time (hours) | % drug dissolved |
|---|---|
| 0.5 | 4 |
| 1 | 6 |
| 2 | 10 |
| 4 | 18 |
| 6 | 25 |
| 8 | 33 |

### EXAMPLE 16

This example illustrates two 600 mg sustained-release tablet formulations of Gabapentin using starch sodium octenyl succinate and Sterotex NF as a release retarding agent. The make up of the composition is illustrated in Table 27.

TABLE 27

| | mg/tablet | |
|---|---|---|
| Ingredients | Formulation A | Formulation B |
| Gabapentin | 600 | 600 |
| Starch sodium octenyl succinate | 200 | 292.5 |
| Emcocel 90M | 25 | — |
| Sterotex NF | 80 | 67.5 |
| PVP K 25 | 35 | 35 |
| Magnesium stearate | 5 | 5 |

The method comprised the following steps:
1. Gabapentin was screened through 850 micron mesh and was granulated with PVP solution (15% w/w in Ethanol).
2. Granules were dried at 45° C. in a tray drier to obtain loss of drying of 1-2% w/w.
3. Starch sodium octenyl succinate, Emcocel 90M, Sterotex NF and magnesium stearate were screened through 355 micron mesh and blended with the granules of step 1.
4. Tablets were compressed using 19×9 mm, capsule shaped punches.

Tablets were tested for dissolution using USP-2 apparatus, paddle speed of 50 rpm and using phosphate buffer pH 6.8, 900 ml as a dissolution media. Results of these tests are recorded in Table 28.

TABLE 28

% drug dissolved

| Time (hours) | Formulation A | Formulation B |
|---|---|---|
| 1 | 25 | 18 |
| 2 | 41 | 31 |
| 3 | 56 | 43 |
| 4 | 68 | 52 |
| 5 | 76 | 65 |
| 6 | 91 | 75 |
| 7 | — | 83 |

### EXAMPLE 17

This example illustrates a 900 mg controlled release Gabapentin formulation using starch sodium octenyl succinate and Sterotex NF as a release retarding agent. The composition's make up is shown in Table 29.

TABLE 29

| Ingredients | mg/tab |
|---|---|
| Gabapentin | 900 |
| PVP K 25 | 52.5 |
| Starch sodium octenyl succinate | 300 |
| Sterotex NF | 120 |
| Emcocel 90M | 37.5 |
| Magnesium stearate | 7.5 |

The method comprised the following steps:
1. Gabapentin was passed through 850 micron mesh and granulated with PVP solution (15% w/w in Ethanol)
2. Granules were dried at 45° C. in a tray drier.
3. Dried granules were sifted through 850 micron mesh and mixed with extragranular material (Starch sodium octenyl succinate, Sterotex, Emcocel and Magnesium stearate passed through 355 micron mesh)
4. Tablets were compressed using 21×10 mm oval punches.

Tablets were tested for dissolution using USP-2 apparatus, paddle speed of 50 rpm and using phosphate buffer pH 6.8, 900 ml as a dissolution media. Results of the dissolution tests are recorded in Table 30.

TABLE 30

| Time (hours) | % drug dissolved |
|---|---|
| 1 | 18 |
| 2 | 32 |
| 3 | 44 |
| 4 | 57 |
| 5 | 67 |
| 6 | 76 |
| 7 | 87 |

### EXAMPLE 18

This example illustrates a 900 mg controlled release Gabapentin formulation using starch sodium octenyl succinate and Sterotex NF as release retarding agent. The composition is recorded in Table 31

TABLE 31

| Ingredients | mg/tab |
|---|---|
| Gabapentin | 900 |
| PVP K 25 | 52.5 |
| Starch sodium octenyl succinate | 175 |
| Sterotex NF | 90 |
| Emcocel 90M | 36.5 |
| Magnesium stearate | 6.0 |

The method comprised the steps of:
1. Gabapentin was passed through 850 micron mesh and granulated with PVP solution (15% w/w in Ethanol)
2. Granules were dried at 45° C. in a tray drier.
3. Dried granules were sifted through 850 micron mesh and mixed with extragranular material (Starch sodium octenyl succinate, Sterotex, Emcocel and Magnesium stearate passed through 355 micron mesh)
4. Tablets were compressed using 21×10 mm oval punches.

Tablets were tested for dissolution using USP-2 apparatus, paddle speed of 50 rpm and using 900 ml 0.06 N HCl as a dissolution media. Results are recorded in Table 32.

TABLE 32

| Time (hours) | % drug dissolved |
|---|---|
| 1 | 27 |
| 2 | 48 |
| 3 | 67 |
| 4 | 80 |
| 6 | 94 |

### EXAMPLE 19

This example illustrates a 900 mg controlled release Gabapentin formulation using starch sodium octenyl succinate and Sterotex NF as a release retarding agent. The composition is recorded in Table 33.

TABLE 33

| Ingredients | mg/tab |
|---|---|
| Gabapentin | 900 |
| PVP K 25 | 52.5 |
| Starch sodium octenyl succinate | 253 |
| Sterotex NF | 200 |
| Emcocel 90M | 37.5 |
| Magnesium stearate | 7.0 |

The method comprised the steps of:

1. Gabapentin was passed through 850 micron mesh and granulated with PVP solution (15% w/w in Ethanol)
2. Granules were dried at 45° C. in a tray drier.
3. Dried granules were sifted through 850 micron mesh and mixed with extragranular material (Starch sodium octenyl succinate, Sterotex, Emcocel and Magnesium stearate passed through 355 micron mesh)
4. Tablets were compressed using 21×10 mm oval punches.

Tablets were tested for dissolution using USP-2 apparatus, paddle speed of 50 rpm and using 900 ml 0.06 N HCl as a dissolution media. Results are recorded in Table 34

TABLE 34

| Time (hours) | % drug dissolved |
|---|---|
| 1 | 20 |
| 2 | 35 |
| 4 | 61 |
| 6 | 79 |
| 8 | 91 |
| 10 | 96 |

### EXAMPLE 20

This example illustrates a sustained-release tablet formulation of Galantamine using starch sodium octenyl succinate as a release retarding agent. The composition is shown in Table 35.

TABLE 35

| Ingredients | mg/tablet |
|---|---|
| Galantamine Hydrobromide | 31 |
| (equivalent to 24 mg base) | |
| Starch Sodium Octenyl Succinate | 319 |
| PVP-K-25 | 24 |
| Emcocel 90M | 52 |
| Cab-O-Sil | 2 |
| Sodium Stearyl Fumarate | 2 |

The method comprised the following steps:
1. Galantamine and Starch Sodium Octenyl Succinate were weighed and screened through 355 micron mesh and thoroughly blended.
2. The powder blend of step-1 was granulated with 20% PVP solution in a mixture of Ethanol and water (70:30).
3. The granules were dried at 60° C. to obtain LOD of 2.5-3.5%.
4. The dried granules were blended with Emcocel, Cab-o-Sil and Sodium stearyl fumarate (screened through 355 micron mesh).
5. Tablets were compressed using 11 mm, round punches.

Tablets were tested for dissolution using USP-2 apparatus, paddle speed of 50 rpm and using 0.06N HCl as a dissolution media for first 2 hours and then changeover to 6.8 Ph phosphate buffer containing Amylase (216 mg/lit) for 2-6 hours. Dissolution results are recorded in Table 36.

TABLE 36

| Time (hours) | % drug dissolved |
|---|---|
| 1 | 23 |
| 2 | 40 |
| 4 | 68 |
| 6 | 102 |

### EXAMPLE 21

This example illustrates a sustained-release tablet formulation of Galantamine using starch sodium octenyl succinate as a release retarding agent. The composition is shown in Table 37.

TABLE 37

| Ingredients | mg/tablet |
|---|---|
| Galantamine Hydrobromide | 31 |
| (equivalent to 24 mg base) | |
| Starch Sodium Octenyl Succinate | 569 |
| PVP-K-25 | 28.4 |
| Cab-O-Sil | 3.1 |
| Sodium stearyl fumarate | 3.1 |

The method comprised the following steps:
1. Galantamine and Starch Sodium Octenyl Succinate were weighed and screened through 355 micron mesh and thoroughly blended.
2. The powder blend of step-1 was granulated with 20% PVP solution in a mixture of Ethanol and water (70:30).
3. The granules were dried at 60° C. to obtain LOD of 2.5-3.5%.
4. The dried granules were blended with Emcocel, Cab-o-Sil and Sodium stearyl fumarate (screened through 355 micron mesh).
5. Tablets were compressed using 18×8.6 mm, capsule shaped punches.

Tablets were tested for dissolution using USP-2 apparatus, paddle speed of 50 rpm and using 0.06N HCl as a dissolution media for first 2 hours and then changeover to 6.8 Ph phosphate buffer containing Amylase (216 mg/lit) for 2-6 hours. Dissolution results are recorded in Table 38.

TABLE 38

| Time (hours) | % drug dissolved |
|---|---|
| 1 | 19 |
| 2 | 32 |
| 4 | 52 |
| 6 | 65 |
| 8 | 78 |
| 10 | 93 |

### EXAMPLE 22

This example illustrates a 500 mg controlled release Ciprofloxacin formulation using starch sodium octenyl succinate and Sterotex NF as a release retarding agent and citric acid as enzyme activity reducing agent. The make up of the composition is set out in Table 39.

TABLE 39

| Ingredients | mg/tab |
|---|---|
| Ciprofloxacin Hydrochloride | 500 |
| Citric acid | 50 |
| Starch sodium octenyl succinate | 300 |
| Sterotex NF | 40 |
| Emcocel 90M | 45 |
| Magnesium stearate | 9- |

The method comprised the following steps:

1. Ciprofloxacin, citric acid, starch sodium octenyl succinate and sterotex NF were passed through 850 micron mesh and blended.
2. The powder of step 1 was slugged using 21 mm round punches.
3. The slugs were passed through 22 mesh to obtain granules.
4. Granules were mixed with emcocel 90M and magnesium stearate.
5. Tablets were compressed using 21×10 mm oval punches.

### EXAMPLE 23

This example illustrates a 120 mg controlled release Propranolol formulation using starch sodium octenyl succinate and Sterotex NF as a release retarding agent and citric acid as enzyme activity reducing agent. The composition was composed as set out in Table 40.

TABLE 40

| Ingredients | mg/tab |
|---|---|
| Propranolol Hydrochloride | 120 |
| Citric acid | 60 |
| Starch sodium octenyl succinate | 250 |
| Sterotex NF | 60 |
| Emcocel 90M | 25 |
| Magnesium stearate | 6.0 |

The method comprised the following steps:
1. Propranolol hydrochloride, citric acid and starch sodium octenyl succinate were passed through 850 micron mesh and granulated with PVP solution (15% w/w in Ethanol)
2. Granules were dried at 45° C. in a tray drier.
3. Dried granules were sifted through 850 micron mesh and mixed with extragranular material-Emcocel and Magnesium stearate
4. Tablets were compressed using 11 m round punches.

1: A controlled or sustained release solid pharmaceutical excipient, comprising a release controlling excipient comprising an amphiphilic starch.

2: An excipient as claimed in claim 1, wherein the amphiphilic starch is an alkyl, alkenyl, aralkyl or aralkenyl succinate or glutarate starch.

3: An excipient as claimed in claim 1, wherein the amphiphilic starch is or includes a C₆ to C₁₆ alkenyl succinate starch.

4: An excipient as claimed in claim 3, wherein the C₆ to C₁₆ alkenyl succinate starch is n-octenyl succinate starch or sodium octenyl succinate starch.

5: An excipient as claimed in claim 1, further comprising at least one oily or fatty component.

6: An excipient as claimed in claim 5, wherein the oily or fatty component is or includes a fatty acid, derivative or salt, a mineral oil, a vegetable oil or a wax.

7: An excipient as claimed in claim 6, wherein the vegetable oil is a hydrogenated vegetable oil.

8: An excipient as claimed in claim 7, wherein the hydrogenated vegetable oil is or includes hydrogenated cottonseed oil, hydrogenated castor oil, hydrogenated palm oil or hydrogenated soybean oil.

9: An excipient as claimed in claim 5, wherein the fatty or oily component is or includes sodium stearyl fumarate, calcium stearate, magnesium stearate, glyceryl monooleate, glyceryl monostearate, glyceryl palmitostearate, medium chain glycerides, mineral oil or stearyl alcohol.

10: An excipient as claimed in claim 5, wherein the at least one oily or fatty component is present in an amount equivalent to up to 40% of the amount of amphiphilic starch in the excipient.

11: An excipient as claimed in claim 1, in a free-flowing powdered or granular form.

12: An excipient as claimed in claim 1, for use in the preparation of a controlled or sustained release solid pharmaceutical composition.

13: An excipient as claimed in claim 12, being sufficiently compressible for use in the formation of tablets by direct compression or by compression of a granulate formed from the excipient.

14: A controlled or sustained release solid pharmaceutical composition, comprising a pharmaceutically active agent and an excipient as claimed in claim 1.

15: A composition as claimed in claim 14, wherein the composition comprises at least 50% active agent by weight.

16: A composition as claimed in claim 15, wherein the composition comprises at least 60, 70 or 80% active agent by weight.

17: A composition as claimed in claim 14, wherein the composition comprises an enzyme activity reducing agent or an enzyme inhibitor.

18: A composition as claimed in claim 17, wherein the enzyme inhibitor is an amylase inhibitor.

19: A composition as claimed in claim 17, wherein the composition includes an acid.

20: A composition as claimed in claim 19, wherein the acid is citric acid, succinic acid, tartaric acid, fumaric acid, maleic acid, lactic acid or ascorbic acid.

21: A composition as claimed in claim 17, wherein the composition includes ascorbic acid, acarbose, phaseolamine, tendaminstat, maltose, maltotriose or nojirimycin.

22: A composition as claimed in claim 14, further comprising a gas-generating agent which reacts with an acid to generate a gas.

23: A composition as claimed in claim 22, wherein the gas-generating agent is sodium bicarbonate or calcium carbonate.

24: A composition as claimed in claim 14, wherein the pharmaceutically active agent is an antiepileptic, antiasthmatic, antiulcer, analgesic, antihypertensive, antibiotic, antipsychotic, anticancer, antimuscarinic, diuretic, antimigraine, antiviral, anti-inflammatory, sedative, antidiabetic, antidepressant, antihistaminic, an antialzheimers drug or a lipid lowering drug.

25: A composition as claimed in claim 24, wherein the active agent is gabapentin, galantamine, topiramate, oxycodone, oxymorphone, hydromorphone or methylphenidate.

26: A composition as claimed in claim 14, wherein the pharmaceutically active agent is present in an amount ranging from 5 to 1200 mg.

27: A composition as claimed in claim 14, wherein the amphiphilic starch comprises from about 2, 5, 7 or 10% to about 80, 85, 90, 95 or 99% by weight of the composition.

28: A composition as claimed in claim 14, comprising an oily or fatty component in an amount from about 2, 5, 7 or 10% to 40, 45, 50, 55 or 60% by weight of the composition, preferably from about 5-20% by weight of the composition.

29: A composition as claimed in claim 14, wherein the composition is in the form of a tablet, a hard gelatin capsule, an extrudate, pellets, a powder, granules, or a suppository.

30: A composition as claimed in claim 29, wherein the composition is in the form of a tablet for ingestion into the gastrointestinal tract.

31: A composition as claimed in claim 14, further comprising a lubricant, a binder, a disintegrating agent, a colouring agent, a flavouring agent, a preservative, a stabiliser, a glidant, a filler, or a bulking agent.

32: A composition as claimed in claim 14, coated with a film of a coating agent.

33: A composition as claimed in claim 32, wherein the coating is substantially unbroken.

34: A composition as claimed in claim 32, wherein the coating comprises a polyvinyl alcohol, a polyacrylate, a polymethacrylate, a cellulose or a cellulose derivative.

35: A method of preparing a controlled or sustained release solid pharmaceutical composition comprising the use of an excipient as claimed in claim 1.

36: A method as claimed in claim 35, wherein the controlled or sustained release solid pharmaceutical composition is a controlled or sustained release solid pharmaceutical composition as claimed in claim 14.

37: A method as claimed in claim 35, comprising directly compressing a mixture comprising the excipient into a controlled or sustained release solid pharmaceutical tablet.

38: A method as claimed in claim 35, comprising forming a granulate comprising the excipient and compressing said granulate into a controlled or sustained release solid pharmaceutical tablet.

39: A method as claimed in claim 35, further comprising the step of coating the tablet.

40: A pharmaceutical composition whenever prepared by a method as claimed in claim 35.

41: A controlled or sustained release gabapentin formulation, comprising from 2, 5, 7 or 10% to 75, 80, 85, 90 or 95% of sodium octenyl succinate starch.

42: A formulation as claimed in claim 41, comprising a pharmaceutically effective amount of gabapentin, about 5, 7, 10 or 15% to 70, 75, 80 or 85% of sodium octenyl succinate starch and about 5, 7, 10 or 15% to 30, 35, 40, 45 or 50% of oily or fatty component by weight of the composition.

43: A controlled or sustained release galantamine formulation, comprising from 2, 5, 7 or 10% to 75, 80, 85, 90 or 95% of sodium octenyl succinate starch.

44: A formulation as claimed in claim 43, comprising a pharmaceutically effective amount of galantamine, and about 65, 70, 75, 80 or 85% of sodium octenyl succinate starch.
